(19) United States
(12) Patent Application Publication
(10) Pub. No.: US 2004/0262192 A1
(43) Pub. Date: Dec. 30, 2004

(54) Chemically resistant vial for cosmetic products

(75) Inventor: Denis Jean-Marie Lhoyer, Senlis (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: SOCIETE GENERALE DE BROSSERIE, Mouy (FR)

(21) Appl. No.: 10/876,564

(22) Filed: Jun. 28, 2004

(30) Foreign Application Priority Data
Jun. 30, 2003 (FR) 0307897

(51) Int. Cl. A61K 6/00

(52) U.S. Cl. 206/581

(57) ABSTRACT

Waterproof mascara is packaged in vials made of polyolefin crosslinked by exposure to ionising radiation and thus made resistant to the solvents contained in the mascara.

The invention relates to a cosmetic product comprising at least one polyolefin vial and a cosmetic composition contained in the vial.

Mascara is usually sold in vials of circular, square or complex cross-section which enable it to be stored and applied.

Two types of mascara formulation are found, namely so-called “cream” formulas and water-resistant formulas known as “waterproof”. Vials for the cream formulas are made of polyolefin, especially low density polyethylene, high density polyethylene, polypropylene, statistical copolymers of propylene. These polymers are advantageous in terms of their manufacturing cost. On the other hand they are not suitable for waterproof formulas as they are degraded by the chemically aggressive organic solvents contained in these compositions. Therefore, polyvinyl chloride vials are used for the latter. However, this polymer is more expensive than the polyolefins and may have carcinogenic effects owing to the presence of chlorine in the molecule. In addition, its corrosive nature makes a protective surface treatment of the mould necessary.

Apart from the drawbacks inherent in PVC, the use of two different materials requires the availability of different moulds for producing vials of the same capacity, as PVC shrinks less on moulding than polyolefins.

The aim of the invention is to overcome these drawbacks.

The invention relates in particular to a cosmetic product of the type defined hereinbefore and provides that the polyolefin is crosslinked.

Crosslinking applied to any one of the polyolefins used for packaging cream mascara confers chemical resistance on it which makes it compatible with waterproof mascara. The crosslinking treatment has virtually no effect on the manufacturing cost of the vials, which means that they can be used for both types of mascara equally, while ensuring health and environmental safety.

Some optional features of the invention, additional or alternative, are stated below:

The crosslinked polyolefin is polyethylene.

The crosslinked polyolefin is high density polyethylene.

The cosmetic composition comprises a chemically aggressive substance.

The chemically aggressive substance is an organic solvent.

The cosmetic composition is waterproof mascara.

The polyolefin is crosslinked by exposure to ionising radiation.

The ionising radiation is beta radiation.

The ionising radiation is gamma radiation.

The ionising radiation is applied at a dose of between 3 and 150 kGy.

The ionising radiation is applied in a dose of between 50 and 120 kGy.

The invention also relates to a process for obtaining a cosmetic product as defined above wherein at least one polyolefin vial and, if appropriate, a collective packaging containing a plurality of such vials, is exposed to said ionising radiation, and the or each vial is filled with said cosmetic composition.

The features and advantages of the invention are disclosed in more detail in the description that follows.

Bombardment with beta or gamma rays triggers the crosslinking of a polyolefin. This bombardment promotes the breaking of the macromolecular chains and hence their reorganisation into a stable saturated three-dimensional network. This crosslinking causes changes in the mechanical, thermal and chemical characteristics.

Tests have shown that it is possible to produce a polyethylene vial crosslinked by ionisation which meets all the packaging requirements for waterproof mascara.

An embodiment of the invention is described below.

High density polyethylene vials are produced, intended for waterproof mascara, which are subjected to all the usual production steps such as decoration, application of varnish, fitting of the squeezer device. The finished vials are assembled in collective packaging such as cardboard boxes which are placed on pallets for transporting. The pallets are then subjected to a source of cobalt 60 generating gamma rays. The emission of gamma radiation crosslinks the vials without the need to unpack them. The dose received is between 3 and 150 kGy. Tests have shown that a dose of between 50 and 120 kGy ensures that the material is resistant to the solvents contained in the mascara.

The vials are then ready to be delivered, for example, to a cosmetic laboratory which will use them for packaging waterproof mascara. Alternatively, the crosslinking operation may be carried out at any intermediate stage in the transformation and handling of the vials.

Similar results are obtained with a bombardment of beta rays but in this case the cardboard boxes have to be removed from the pallets because of the lesser penetration of these rays.

1. Cosmetic product comprising at least one polyolefin vial and a cosmetic composition contained in the vial, characterised in that the polyolefin is crosslinked.

2. Cosmetic product according to claim 1, wherein the crosslinked polyolefin is polyethylene.

3. Cosmetic product according to claim 1, wherein the crosslinked polyolefin is high density polyethylene.

4. Cosmetic product according to one of the preceding claims, wherein the cosmetic composition comprises a chemically aggressive substance.

5. Cosmetic product according to claim 4, wherein said chemically aggressive substance is an organic solvent.

6. Cosmetic product according to one of the preceding claims, wherein said cosmetic composition is waterproof mascara.

7. Cosmetic product according to one of the preceding claims, wherein said polyolefin is crosslinked by exposure to ionising radiation.

8. Cosmetic product according to claim 7, wherein said ionising radiation is beta radiation.

9. Cosmetic product according to claim 7, wherein said ionising radiation is gamma radiation.

10. Cosmetic product according to claim 9, wherein said ionising radiation is applied in a dose of between 3 and 150 kGy.

11. Cosmetic product according to claim 9, wherein said ionising radiation is applied in a dose of between 50 and 120 kGy.

12. Process for obtaining a cosmetic product according to one of claims 7 to 11, wherein at least one polyolefin vial is exposed to said ionising radiation and said vial is filled with said cosmetic composition.

13. Process according to claim 12, wherein a collective packaging containing a plurality of vials is exposed to said ionising radiation.
